(19) United States
(12) Patent Application Publication
(10) Pub. No.: US 2026/0297831 A1
(43) Pub. Date: Oct. 1, 2026

(54) TEMPERATURE-SPECIFIC PERFORMANCE DEVIATION CORRECTION APPARATUS AND CORRECTION METHOD FOR PHOTOSENSOR DETECTING NEPHELOMETRIC TURBIDITY UNITS

(71) Applicant: LG ELECTRONICS INC., Seoul (KR)

(72) Inventors: Hosik KANG, Seoul (KR); Wanseop CHOI, Seoul (KR); Hyunchul KIM, Seoul (KR)

(73) Assignee: LG ELECTRONICS INC., Seoul (KR)

(21) Appl. No.: 18/998,111

(22) PCT Filed: Jul. 25, 2022
(86) PCT No.: PCT/KR2022/010901

§ 371 Date: Jan. 23, 2025

(51) Int. Cl. D06F 34/24; G01N 21/88; G01N 21/93; G01N 21/94; G01N 33/18

(52) U.S. Cl. CPC D06F 34/24; G01N 21/8806; G01N 21/93; G01N 21/94; G01N 33/18; G01N 2201/1211

(57) ABSTRACT

The present invention relates to an apparatus for correcting temperature-specific performance deviation of a photosensor, comprising a sensor, which includes a plurality of photosensors, and a controller, which: detects, by means of each of the plurality of photosensors, nephelometric turbidity unit (NTU) values at a first temperature and a second temperature; detects the difference between the NTU values according to the first temperature and the second temperature; calculates a temperature-specific NTU value difference estimate for each photosensor on the basis of the detected NTU value difference; calculates temperature-specific NTU value correction values for each of the plurality of photosensors; and corrects the NTU values of each of the plurality of photosensors according to the calculated temperature-specific NTU value correction values.

### TECHNICAL FIELD

The present disclosure relates to a photosensor that detects a turbidity of a solution to detect a water quality in a washing machine or the like.

### BACKGROUND ART

In recent years, with the advancement of technologies, automated washing machines have emerged that automatically detect an amount of contamination and a degree of rinsing of laundry, and automatically perform a designated function based on the detected amount of contamination and the detected degree of rinsing. For the operation of such an automated washing machine, it is essential to accurately detect a water quality of a solution, that is, water in which the laundry is accommodated.

In order to detect the water quality of the solution, various sensors are used. A sensor typically used to detect the water quality can be a photosensor. In this case, the photosensor can emit light to a solution, and detect a concentration of suspended matter, that is, a turbidity, of the solution based on a difference between an amount of the emitted light and an amount of light transmitted or reflected by the solution. Furthermore, the turbidity (nephelometric turbidity unit, NTU) detected through a photosensor in this manner can be a criterion for determining the water quality of the solution.

However, in the case of the photosensor, the detected light sensitivity can vary significantly depending on the temperature characteristics of an optical device provided in the photosensor. In this case, if the light sensitivity changes, a value of turbidity detected by the photosensor can vary significantly. Depending on the temperature characteristics of the optical device, there is a performance deviation by temperature, and thus there is a problem in that a value of turbidity detected by a photosensor can vary greatly depending on the temperature of the solution due to the temperature-specific performance deviation of the optical device.

Meanwhile, the temperature characteristics of the optical device are determined during the manufacture of the optical device, and even if the same type of sensor is manufactured by the same manufacturer, the temperature-specific performance deviation can be different. Moreover, there is a problem in that such a temperature-specific performance deviation for a photosensor increases as the temperature of the solution increases.

In order to solve such a problem of the temperature-specific performance deviation for the optical device, there has been introduced a method of providing a plurality of photosensors, calculating an average value of the temperature-specific performance deviations of the plurality of photosensors, and compensating for the performance deviation based on the calculated average value. However, the method of compensation based on an average value of temperature-specific performance deviation has a problem in that it cannot compensate for an individual deviation of each photosensor.

That is, since the individual deviation of the photosensor cannot be corrected, the turbidity detection performance of each of the plurality of photosensors varies with temperature, and therefore, there is a problem in that a difference in turbidity (NTU) detected by each photosensor increases depending on the temperature due to the different detection performance. Therefore, there is a problem in that the accuracy of the detected turbidity can decrease significantly as the temperature increases.

### DISCLOSURE OF INVENTION

### Technical Problem

The present disclosure aims to solve the foregoing problems and other problems, and an aspect of the present disclosure is to provide a temperature-specific performance deviation correction apparatus for a photosensor capable of minimizing a deviation in turbidity detected by temperature for each of a plurality of photosensors, and a correction method thereof.

### Solution to Problem

In order to achieve the foregoing or other objectives, according to an aspect of the present disclosure, temperature-specific performance deviation correction apparatus for a photosensor according to an embodiment of the present disclosure can include a sensing part comprising a plurality of photosensors, and a control part that detects turbidity values at a first temperature and a second temperature for a test solution having a same turbidity from each of the plurality of photosensors, detects a difference between the turbidity values according to the first temperature and the second temperature, calculates temperature-specific turbidity value (NTU) difference estimates for each photosensor based on the detected turbidity value difference, calculates temperature-specific turbidity value correction values for each of the plurality of photosensors by reflecting the temperature-specific turbidity value difference estimates to temperature-specific measurement values measured from each of the plurality of photosensors at each temperature for the test solution having the same turbidity, and corrects the turbidity values of each of the plurality of photosensors according to the calculated temperature-specific turbidity value correction values.

In one embodiment, the control part can calculate temperature-specific turbidity value difference estimates for the each photosensor based on a deviation relation that uses a difference between turbidity values according to the first temperature and the second temperature according to the following Equation 1:

$$NTU difference estimate = \frac{Z \times temperature}{30} - \frac{2 \times Z}{3} \qquad [ Equation 1 ]$$

Here, Z is a difference between a first turbidity value detected at the first temperature and a second turbidity value detected at the second temperature.

In one embodiment, the first temperature and the second temperature can have a temperature difference between 25 degrees and 30 degrees.

In one embodiment, the test solution can be a solution whose turbidity does not change according to a temperature change.

In one embodiment, the control part can detect an NTU conversion equation that converts a detection value of a photosensor into a turbidity value (NTU) based on turbidity values detected from the test solution having different turbidities at the same temperature, and detect a difference between a first conversion value obtained by converting a first detection value of a photosensor detected when the test solution is at the first temperature at a same turbidity according to the detected conversion equation and a second conversion value obtained by converting a second detection value of the photosensor detected when the test solution is at the second temperature at the same turbidity according to the detected conversion equation, as a difference between turbidity values according to the first temperature and the second temperature.

In one embodiment, the apparatus can further include an accommodation part provided with the plurality of photosensors on an inside thereof to accommodate the test solution, wherein the accommodation part further includes a heating part capable of heating the test solution.

In addition, a washing machine according to an embodiment of the present disclosure can include a sensing part comprising a plurality of photosensors, and a control part that detects turbidities at a first temperature and a second temperature for a test solution having a same turbidity for each of the plurality of photosensors, detects a turbidity difference according to the first temperature and the second temperature based on a result of the detected turbidities, calculates temperature-specific turbidity value (NTU) difference estimates for each photosensor based on the detected turbidity difference, calculates temperature-specific turbidity value correction values for each of the plurality of photosensors by reflecting the temperature-specific turbidity value difference estimates to temperature-specific measurement values measured from each of the plurality of photosensors at each temperature for the test solution having the same turbidity, and corrects the turbidity values of each of the plurality of photosensors according to the calculated temperature-specific turbidity value correction values.

In one embodiment, the control part can calculate temperature-specific turbidity value difference estimates for the each photosensor based on a deviation relation that uses a difference between turbidity values according to the first temperature and the second temperature according to the following Equation 1, wherein the first temperature and the second temperature have a temperature difference between 25 degrees and 30 degrees.

$$NTU difference estimate = \frac{Z \times temperature}{30} - \frac{2 \times Z}{3} \qquad [ Equation 1 ]$$

Here, Z is a difference between a first turbidity value detected at the first temperature and a second turbidity value detected at the second temperature.

In one embodiment, the washing machine can further include a laundry accommodation part provided with the plurality of photosensors on an inside thereof to accommodate the test solution.

In addition, a correction method of correcting temperature-specific performance deviations of a plurality of photosensors for detecting a turbidity of a solution can include detecting, for each of the plurality of photosensors, turbidity values at a first temperature and a second temperature for a test solution having a same turbidity, calculating a difference between the detected turbidity values, calculating temperature-specific turbidity value (NTU) difference estimates for each photosensor based on the calculated difference between the turbidity values, measuring, for the test solution having the same turbidity, turbidity values from each of the plurality of photosensors at each temperature, calculating, for each of the plurality of photosensors, temperature-specific turbidity value correction values by reflecting the temperature-specific turbidity value difference estimates to the turbidity values measured at each temperature, and correcting turbidity values of each of the plurality of photosensors according to the calculated temperature-specific turbidity value correction values.

In one embodiment, the detecting of turbidity values at the first temperature and the second temperature can include detecting turbidity values detected from the test solutions having different turbidities at a same temperature, detecting an NTU conversion equation that converts a detection value of a photosensor into a turbidity value (NTU) based on turbidity values detected from the test solutions having different turbidities, detecting a first conversion value obtained by converting a first detection value of each photosensor detected when the test solution is at the first temperature at a same turbidity according to the detected conversion equation, detecting a second conversion value obtained by converting a second detection value of each photosensor detected when the test solution is at the second temperature at the same turbidity according to the detected conversion equation, and detecting, for each of the plurality of photosensors, a difference between the first conversion value and the second conversion value as a difference between turbidity values according to the first temperature and the second temperature.

In one embodiment, the detecting of a difference between the first conversion value and the second conversion value as a difference between the turbidity values can include detecting photosensors, from among the plurality of photosensors, having a difference between the first conversion value and the second conversion value within a preset range, and excluding the detected photosensors from those for temperature-specific turbidity value correction.

In one embodiment, the calculating of the temperature-specific turbidity value (NTU) difference estimates can include calculating temperature-specific turbidity value difference estimates for the each photosensor based on a deviation relation that uses a difference between turbidity values according to the first temperature and the second temperature according to the following Equation 1:

$$NTU difference estimate = \frac{Z \times temperature}{30} - \frac{2 \times Z}{3} \qquad [ Equation 1 ]$$

Here, Z is a difference between a first turbidity value detected at the first temperature and a second turbidity value detected at the second temperature.

In one embodiment, the first temperature and the second temperature can have a temperature difference between 25 degrees and 30 degrees.

In one embodiment, the test solution can be a solution whose turbidity does not change according to a temperature change.

### Advantageous Effects of Invention

A temperature-specific performance deviation correction apparatus for a photosensor according to the present disclosure and a correction method thereof will be described as follows. According to at least one of embodiments of the present disclosure, the present disclosure can compensate for a turbidity detection performance deviation according to a temperature based on a temperature-specific compensation value calculated for each photosensor, thereby having an effect of reducing a temperature-specific performance deviation for each of a plurality of photosensors.

Therefore, the turbidity detection performance of the plurality of photosensors can change in an identical or similar manner with temperature, thereby having an effect of significantly improving the detection accuracy of a turbidity through a compensation based on a common compensation value (e.g., a compensation by an average value) for the plurality of photosensors.

### BRIEF DESCRIPTION OF DRAWINGS

FIG. 1 is a block diagram showing a structure of a photosensor temperature-specific performance deviation correction apparatus according to an embodiment of the present disclosure.

FIG. 2A is a flowchart showing an operation process of a photosensor temperature-specific performance deviation correction apparatus according to an embodiment of the present disclosure.

FIG. 2B is a flowchart showing in more detail a calibration process for a selected sensor during an operation process of a photosensor temperature-specific performance deviation correction apparatus according to an embodiment of the present disclosure.

FIG. 3 is exemplary diagrams showing an example of a difference in turbidity detection performance according to an increase in temperature for a plurality of photosensors and a difference in turbidity detection performance deviation between different photosensors.

FIG. 4 is exemplary diagrams showing temperature-specific performance deviations of photosensors in a typical case and in a case where the performance deviations are corrected according to an embodiment of the present disclosure.

### MODE FOR THE INVENTION

It should be noted that the technical terms used herein are merely used to describe a specific embodiment, but are not intended to limit the present disclosure. In addition, a singular expression used herein can include a plural expression unless clearly defined otherwise in the context. A suffix “module” or “part” used for elements disclosed in the following description is merely intended for easy description of the specification, and the suffix itself is not intended to have any special meaning or function.

As used herein, terms such as “comprise” or “include” should not be construed to necessarily include all elements or steps described herein, and should be construed not to include some elements or some steps thereof, or should be construed to further include additional elements or steps.

In addition, in describing technologies disclosed herein, when it is determined that a detailed description of known technologies related thereto can unnecessarily obscure the subject matter disclosed herein, the detailed description will be omitted.

Furthermore, the accompanying drawings are provided only for a better understanding of the embodiments disclosed in this specification and are not intended to limit technical concepts disclosed in this specification, and therefore, it should be understood that the accompanying drawings include all modifications, equivalents and substitutes within the concept and technical scope of the present disclosure. In addition, not only respective embodiments described below, but also combinations of embodiments can of course be included within the concept and technical scope of the present disclosure as modifications, equivalents or substitutes.

Hereinafter, the configuration and operation of the present disclosure will be described in more detail with reference to a number of drawings related to the present disclosure.

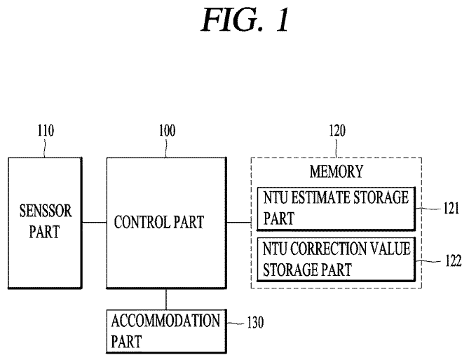

FIG. 1 is a block diagram showing a structure of a photosensor temperature-specific performance deviation correction apparatus 1 according to an embodiment of the present disclosure.

Referring to FIG. 1, a photosensor temperature-specific performance deviation correction apparatus 1 according to an embodiment of the present disclosure can include a sensor part 110 including a plurality of photosensors for detecting turbidity, an accommodation part 130 in which a test solution for measuring turbidity (NTU) values of respective photosensors provided in the sensor part 110 is accommodated, a memory 120, and a control part 100. The elements shown in FIG. 1 are not essential for implementing the photosensor temperature-specific performance deviation correction apparatus 1, and thus the photosensor temperature-specific performance deviation correction apparatus 1 described herein can have more or fewer elements than those listed above.

First, the sensor part 110 can include a plurality of photosensors. The plurality of photosensors can be sensors provided at different positions in an accommodation part (not shown) in which laundry is accommodated, and at least some of the plurality of photosensors can be positioned at positions facing each other. In addition, the accommodation part can accommodate a solution such as water, and when the solution is accommodated in the accommodation part, the plurality of photosensors can be configured to detect light that passes through the solution or is reflected from the solution. To this end, each of the plurality of photosensors can include a light detection part (not shown) that detects light and a light irradiation part (not shown) that can irradiate light.

The turbidity of the solution can be detected based on the light detection results of the plurality of photosensors of the sensor part 110. In this case, the sensor part 110 can convert a turbidity detection result of the solution detected by each photosensor into a digital value and output the converted digital value as a turbidity detection value (turbidity (NTU) value). For this purpose, the sensor part 110 can be provided with at least one analog digital converter (ADC) (not shown).

Furthermore, the photosensor temperature-specific performance deviation correction apparatus 1 can include an accommodation part 130 that accommodates a test solution to detect a turbidity value from each photosensor of the sensor part 110 through an actual measurement. The accommodation part 130 can include a container capable of accommodating the test solution, and the plurality of photosensors can be arranged on an inner surface of the container so as to be disposed so as to measure a turbidity of the test solution when the test solution is accommodated in the container.

Meanwhile, the photosensor temperature-specific performance deviation correction apparatus 1 can include a calibration part (not shown) that can correct a turbidity detection value of each photosensor of the sensor part 110 according to a temperature. The calibration part can correct a turbidity detection value calculated from each of the plurality of photosensors according to an average of change amounts in the turbidity detection values of the plurality of photosensors at each temperature. Here, the average of the temperature-specific turbidity detection value change amounts for the plurality of photosensors can be a value detected in advance by a manufacturer of the sensor part 110 or a value set by the manufacturer.

The calibration part can of course be implemented as an integral part with the control part 100. That is, the control part 100 can of course receive turbidity detection values of each photosensor from the sensor part 110, and collectively calibrate the received turbidity detection values by reflecting the average of the temperature-specific turbidity detection values of the plurality of photosensors stored in the memory 120 to each of the received values. In this case, the control part 100 can also perform a function of the calibration part.

Furthermore, the memory 120 can store data that supports a function of the photosensor temperature-specific performance deviation correction apparatus 1 according to an embodiment of the present disclosure. The memory 120 can store an application or an application program run by the photosensor temperature-specific performance deviation correction apparatus 1 and data and instructions for the operation of the photosensor temperature-specific performance deviation correction apparatus 1.

For example, the memory 120 can store temperature-specific turbidity (NTU) estimates calculated by the control part 100 based on turbidity values calculated at specific temperatures for each of the plurality of photosensors, according to an embodiment of the present disclosure. In addition, turbidity correction values for each of the plurality of photosensors can be stored, which are calculated by the temperature-specific turbidity estimates and the turbidity detection values of each of the plurality of photosensors measured at each temperature.

Hereinafter, an area on the memory 120 in which temperature-specific turbidity estimates for each of the plurality of photosensors are stored is referred to as a turbidity (NTU) estimate storage part 121. Furthermore, an area on the memory 120 in which turbidity correction values for each of the plurality of photosensors are stored is referred to as a turbidity (NTU) correction value storage part 122.

Meanwhile, the control part 100 cab control an overall function of the photosensor temperature-specific performance deviation correction apparatus 1 according to an embodiment of the present disclosure, and can control connected elements. For example, the control part 100 can detect calibrated turbidity detection values corresponding to specific temperatures for any one of the plurality of photosensors. Furthermore, based on a difference between the detected turbidity detection values and a preset temperature-specific deviation relation, temperature-specific turbidity estimates for the any one of the photosensors can be calculated. Furthermore, by measuring turbidities at different temperatures from the any one of the photosensors, and reflecting turbidity estimates to the measured values at different temperatures, temperature-specific turbidity correction values for individual temperature characteristics of the any one of the photosensors can be calculated. Furthermore, the foregoing process can be performed for each of the plurality of photosensors.

Furthermore, the control part 100 can compensate for the turbidity detection value of each of the plurality of photosensors according to the temperature-specific turbidity correction values for each of the plurality of photosensors. Therefore, temperature-specific performance deviations can be individually compensated for each of the plurality of photosensors.

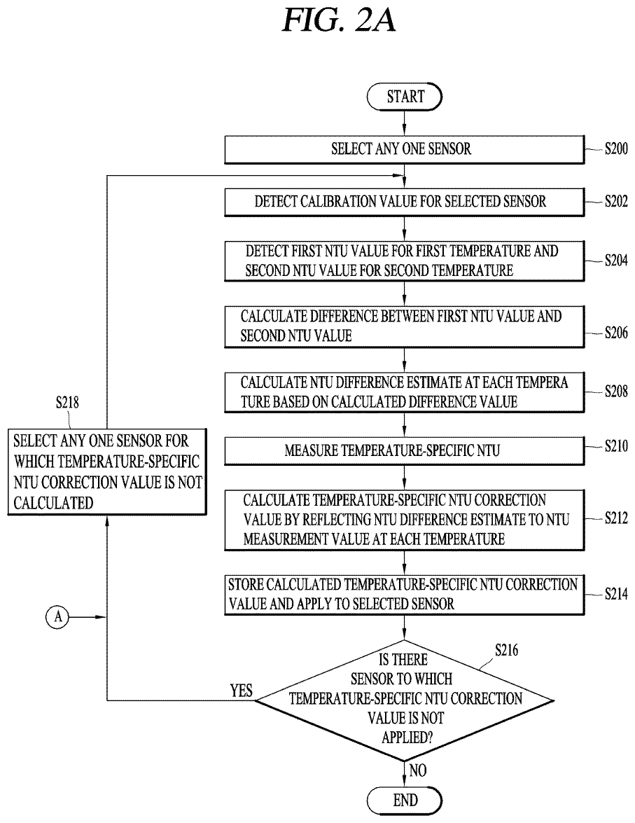

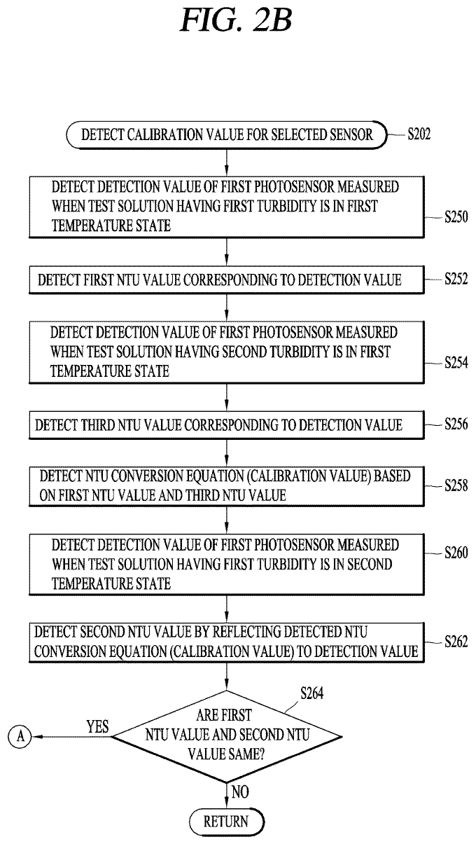

Meanwhile, FIG. 2A is a flowchart showing an operation process of the photosensor temperature-specific performance deviation correction apparatus 1 according to an embodiment of the present disclosure.

Referring to FIG. 2A, the control part 100 of the photosensor temperature-specific performance deviation correction apparatus 1 can select any one of the photosensors of the sensor part 110 (S200). In this case, the control part 100 can select one photosensor (first photosensor) according to a preset sequence.

When the first photosensor is selected, the control part 100 can detect a calibration value for a temperature-specific turbidity detection value based on an average of temperature-specific turbidity detection value change amounts for a plurality of photosensors, that is, a calibration value, for the selected photosensor (S202). Here, the calibration value, which is provided to convert a detection value detected by the photosensor, that is, an analog-to-digital conversion value, into a turbidity detection value (NTU value), can include a conversion formula (NTU conversion equation) for converting the detection value of the photosensor into an NTU value.

Meanwhile, the control part 100 can receive a turbidity detection result that varies with temperature from the first photosensor based on a test solution whose turbidity does not change according to the temperature. In this case, the turbidity detection result can reflect the calibration value.

In this case, a solution whose turbidity does not change with temperature can be used, such as tap water or deionized (DI) water, which is a solution whose turbidity is known in advance. Here, the turbidity of tap water or deionized water can be a solution having a value of 0 NTU.

Meanwhile, the photosensor temperature-specific performance deviation correction apparatus 1 according to an embodiment of the present disclosure can be a washing machine. Furthermore, the washing machine, which is a pre-sale product, can be in a state of testing for tuning and performance test at the manufacturing plant for sale. That is, the control part 100 of the photosensor temperature-specific performance deviation correction apparatus 1 according to an embodiment of the present disclosure can be a control part of the washing machine, and the memory 120 can be a memory provided in the washing machine. Furthermore, the operation process shown in FIG. 2 can be an operation process carried out during the tuning and performance test process of the washing machine. In this case, the accommodation part 130 can be a laundry accommodation part for accommodating laundry in the washing machine, and the plurality of photosensors can be some of the sensors provided in the laundry accommodation part.

In order to receive a turbidity detection result that varies with temperature from the first photosensor, the control part 100 can first detect a turbidity (NTU) value (first NTU value) of the test solution detected by the first photosensor in a first temperature state. Furthermore, for the test solution having a second temperature higher than the standard temperature, a turbidity value (second NTU value) of the test solution detected by the first photosensor can be detected (S206).

Here, the first temperature can be a standard temperature (room temperature of 20 degrees). Furthermore, the second temperature can be a temperature having a preset temperature difference from the first temperature. As an example, the preset temperature difference can be 25 to 30 degrees. The temperature difference can be an optimal temperature difference determined through a plurality of experiments conducted in association with an embodiment of the present disclosure to detect a temperature deviation of a photosensor.

Meanwhile, in the accommodation part 130, subsequent to detecting a first turbidity value, the test solution can be replaced with a test solution heated to a second temperature to detect a second turbidity value. Alternatively, the accommodation part 130 of the photosensor temperature-specific performance deviation correction apparatus 1 according to an embodiment of the present disclosure can further include a heating part (not shown) for heating the accommodated test solution. In this case, the second turbidity value can be detected through heating the accommodation part 130 without replacing the test solution.

Table 1 below shows examples of a first turbidity value detected by a first photosensor at a first temperature, which is a standard temperature (20 degrees), and a second turbidity value detected at a second temperature, which is 50 degrees, for a test solution (tap water) having 0 NTU according to an embodiment of the present disclosure.

TABLE 1

| Temperature | Detected NTU |
|---|---|
| 20 degrees | −4.3 |
| 50 degrees | −30.3 |

In this case, the first turbidity value can be −4.3, and the second turbidity value can be −30.3. That is, in the case of the first photosensor, it can be seen that the control part 100 detects a turbidity of −4.3 NTU at 20 degrees and a turbidity of −30.3 NTU at 50 degrees for tap water having 0 NTU as a result of applying a calibration value (NTU conversion equation) to a detection value (ADC conversion value) of the sensor.

In this case, when the temperature characteristics of the first photosensor do not follow an average change amount in turbidity detection performance according to a temperature, the turbidity value can vary significantly due to a change in turbidity detection performance according to the temperature. Therefore, at 50 degrees, a value that is far from an actual turbidity of 0 NTU can be detected.

Therefore, the control part 100 can calculate a difference between the first turbidity value and the second turbidity value to calculate a turbidity detection temperature deviation of the first photosensor (S206). Thus, in the case of the first photosensor showing the result as in Table 1 above, a difference (Z) between the first turbidity value and the second turbidity value can have a value of −26.

Then, the control part 100 can calculate NTU difference estimates at each temperature for the first photosensor based on a deviation relation as shown in the following Equation 1 according to a difference (Z) between the turbidity values (S208).

$$NTU difference estimate = \frac{Z \times temperature}{30} - \frac{2 \times Z}{3} \qquad [ Equation 1 ]$$

Here, Z is a difference between first and second turbidity values.

In this case, the control part 100 can calculate NTU value estimates for the first photosensor at different temperatures according to the Equation 1. In this case, for a test solution having the value of 0 NTU and whose turbidity does not change with temperature, when the temperature of the test solution changes from 20 degrees to 80 degrees, a result of calculating NTU value estimates detected by the first photosensor according to the deviation relation (Equation 1) can be calculated as shown in Table 2 below.

TABLE 2

| Temperature | NTU difference estimate |
|---|---|
| 20 | −0.0 |
| 30 | −8.7 |
| 40 | −17.4 |
| 50 | −26.0 |
| 60 | −34.7 |
| 70 | −43.4 |
| 80 | −52.1 |

### <Unit Temperature: 10 Degrees>

Then, the control part 100 can measure an NTU value detected at each temperature for the first photosensor (S210). To this end, the control part 100 can heat a test solution accommodated in the accommodation part 130, and detect turbidity (NTU) values detected by the first photosensor whenever the test solution reaches a specific temperature. In this case, the turbidity values of the first photosensor measured at the different temperatures can be values to which a calibration value of the first photosensor is reflected from the detection value (ADC conversion value) of the first photosensor.

TABLE 3

| Temperature | NTU difference estimate |
|---|---|
| 20 | −4.3 |
| 30 | −11.0 |
| 40 | −18.9 |
| 50 | −30.3 |
| 60 | −41.4 |
| 70 | −53.7 |
| 80 | −54.6 |

### <Unit Temperature: 10 Degrees>

Then, the control part 100 can calculate temperature-specific NTU correction values for the first photosensor by reflecting a temperature-specific NTU estimate calculated in the step S208 to temperature-specific NTU measurement values measured as shown in Table 3 above (S212). That is, the control part 100 can calculate NTU correction values corresponding to each temperature by subtracting an NTU estimate at a temperature corresponding to each of the temperature-specific NTU measurement values measured in the step S210. In this case, the temperature-specific NTU correction values for the first photosensor calculated at each temperature from 20 degrees to 80 degrees as shown in the tables above are as shown in Table 4 below.

TABLE 4

| Temper- | NTU measurement | NTU difference | NTU correction |
|---|---|---|---|
| ature | value | estimate | value |
| 20 | −4.3 | −0.0 | −4.3 |
| 30 | −11.0 | −8.7 | −2.3 |
| 40 | −18.9 | −17.4 | −1.6 |
| 50 | −30.3 | −26.0 | −4.3 |
| 60 | −41.4 | −34.7 | −6.7 |
| 70 | −53.7 | −43.4 | −10.3 |
| 80 | −54.6 | −52.1 | −2.5 |

As shown in Table 4 above, when NTU correction values are calculated at different temperatures for the first photosensor, the control part 100 can store the calculated NTU correction values in the memory 120. Furthermore, the stored NTU correction values can be applied to the currently selected sensor, that is, the first photosensor (S214). Therefore, the control part 100 can output an NTU value detected by the first photosensor according to a preset calibration value (NTU conversion equation) as a turbidity value detected by the first photosensor by correcting it based on the NTU correction value set at each temperature.

Accordingly, even when the temperature characteristics of the first photosensor do not follow an average change amount in turbidity detection performance according to a temperature, it can be corrected to follow the average change amount in the turbidity detection performance according to the temperature based on a temperature-specific NTU correction value calculated for the first photosensor. That is, the individual temperature characteristics of the first photosensor can be corrected according to the average temperature characteristics.

Then, the control part 100 can check whether there is a photosensor for which the temperature-specific NTU correction value has not been calculated and applied (S216). Furthermore, if there is at least one sensor for which a temperature-specific NTU correction value is not calculated and applied, then any one of the at least one sensor can be selected as the first photosensor (S218). Then, the process can proceed to step S202 to perform the process from step S202 to step S214 for the selected first photosensor.

On the contrary, if there is no photosensor for which the temperature-specific NTU correction value is not calculated and applied as a result of the check in the step S216, then it is determined that the temperature-specific NTU correction value has been calculated and applied to all photosensors, and the operation process of FIG. 2A for correcting the temperature-specific performance deviation of the photosensor can be terminated.

Meanwhile, the photosensor temperature-specific performance deviation correction apparatus 1 according to an embodiment of the present disclosure can calculate, based on turbidity (NTU) values of a first photosensor detected from test solutions having different turbidities, a calibration value of the first photosensor, and determine whether the temperature characteristics of the first photosensor follow an average change amount of turbidity detection performance according to a temperature based on the calculated calibration value. Furthermore, if the temperature characteristics of the first photosensor as a result of the determination follow the average change amount of the turbidity detection performance according to the temperature, the NTU correction value according to the operation process of FIG. 2B cannot be calculated.

FIG. 2B is a flowchart showing an operation process of calculating a calibration value for a currently selected first photosensor and determining whether the temperature characteristics of the first photosensor follow an average change amount of turbidity detection performance according to a temperature based on the calculated calibration value during the operation process of the photosensor temperature-specific performance deviation correction apparatus 1 according to an embodiment of the present disclosure. In this case, the calibration value can be calculated based on a turbidity detection result of the photosensor for test solutions with different turbidities at the same temperature.

Referring to FIG. 2B, when any one photosensor (first photosensor) is selected in the step S200 of FIG. 2A, the control part 100 can detect a detection value of the selected photosensor (first photosensor) for a test solution having a preset first turbidity (S250).

Here, the test solution can be a solution whose turbidity does not change with temperature as described above. As an example, the test solution can be tap water, in which case the first turbidity can be 0 NTU. Furthermore, the test solution can be in a standard temperature (20 degrees), that is, a first temperature state.

Additionally, a detection value of the photosensor can refer to a detection value before being converted into an NTU value. That is, it can be a value of an analog-to-digital converter before being converted into an NTU value by the NTU conversion equation. In this case, the NTU conversion equation can include a calibration value for calibration. That is, the NTU conversion equation can be provided to convert a value of the analog-to-digital converter of the photosensor into an NTU value by reflecting a preset calibration value. Furthermore, the control part 100 can convert a detection result of a test solution having a first turbidity (e.g., 0 NTU) in the first temperature (e.g., standard temperature, 20 degrees) state into an NTU value based on the NTU conversion equation set for the first photosensor (S252). That is, for a test solution having a first turbidity in the first temperature state, an NTU value (first NTU value) of the first photosensor to which the calibration value is reflected can be detected.

Furthermore, the control part 100 can detect a detection value of the first photosensor for a test solution having a different turbidity (second turbidity) from the first turbidity at the same temperature (S254). For example, when the first turbidity is 0 NTU as described above, the second turbidity can be 1000 NTU. For this purpose, the test solution in the accommodation part 130 can be replaced with a solution having a different turbidity. Alternatively, an additive for changing the turbidity of the test solution can be added to the test solution in an amount necessary for changing to the second turbidity.

When the turbidity of the test solution is changed to the second turbidity, the control part 100 can receive a detection value of the first photosensor for the test solution having the second turbidity (1000 NTU) at the same temperature (first temperature) from the sensor part 110. Furthermore, the detection value of the first photosensor can be converted into an NTU value based on the NTU conversion equation set for the first photosensor (S256). That is, for a test solution having a second turbidity in the first temperature state, an NTU value (third NTU value) of the first photosensor to which the calibration value is reflected can be detected.

Furthermore, the control part 100 can detect an NTU conversion equation for the first photosensor based on the first NTU value and the third NTU value (S258).

When the NTU conversion equation for the first photosensor is detected, the control part 100 can detect the detection values of the first photosensor for test solutions at different temperatures for the same turbidity (S260). For example, the test solution accommodated in the accommodation part 130 can be replaced with a test solution having a first turbidity (0 NTU). Furthermore, the accommodation part 130 can be heated to form a test solution having the first turbidity in the second temperature (e.g., 50 degrees) state.

In step S260, when a detection value of the first photosensor for a test solution having the same turbidity but whose temperature has increased to a second temperature is detected, the control part 100 can convert the detected detection value into an NTU value (second NTU value) according to the NTU conversion equation detected in the step S258 (S262). Furthermore, the first NTU value and the second NTU value can be compared to check whether they are the same (S264).

In this case, when the first NTU value and the second NTU value are the same (e.g., 0 NTU), or when a difference between the first NTU value and the second NTU value is within a preset range, it can be determined that the first photosensor detects the same turbidity despite the temperature change. That is, it can be determined that a turbidity performance detection deviation according to a temperature is compensated based on the NTU conversion equation. In this case, the NTU conversion equation can be provided to compensate for an average change amount for each temperature characteristic of a plurality of photosensors. Thus, the temperature characteristics of the first photosensor can follow an average change amount for each temperature characteristic of the plurality of photosensors.

Therefore, the control part 100 can determine that individual correction for the first photosensor is not necessary. In this case, the control part 100 does not proceed to step S204 in FIG. 2A, but proceeds directly to step S218, so as to re-select another photosensor for which a temperature-specific NTU correction value has not been calculated as the first photosensor. Furthermore, based on the newly selected first photosensor, the control part can proceed again to the step S202 in FIG. 2A. In this case, the step S250 of FIG. 2B can be performed again.

On the contrary, if a result of the check in the step S264 shows that the first NTU value and the second NTU value are not the same or a difference between the first NTU value and the second NTU value is out of the preset range, then the control part 100 can determine that the temperature characteristics of the first photosensor do not follow an average change amount of temperature-specific turbidity detection performance for the plurality of photosensors. Therefore, the control part 100 can proceed to step S204 in FIG. 2A to detect the first NTU value and the second NTU value and calculate a difference between the NTU values detected in step S206 in FIG. 2A. In this case, the first NTU value and the second NTU value can be used as they are as calculated during the operation process of FIG. 2B.

Meanwhile, in the description of the FIGS. 2A and 2B, a configuration in which the control part 100 sequentially selects a plurality of photosensors one by one and calculates a temperature-specific NTU correction value for the selected photosensor has been described as an example, but the control part 100 can of course select a plurality of photosensors simultaneously and perform the configuration of the FIGS. 2A and 2B in parallel for each of the selected plurality of photosensors.

In this case, the process of detecting the first turbidity value for the test solution having the first turbidity and being in the first temperature state (step S250 of FIG. 2B), the process of detecting the third turbidity value for the test solution having the second turbidity and being in the first temperature state (step S254 of FIG. 2B), and the process of detecting the second turbidity value for the test solution having the first turbidity and being in the second temperature state (step S262 of FIG. 2B) can be shared with one another. That is, when the process of FIG. 2B is carried out in parallel for each of the plurality of photosensors, turbidity values (first, third, and second turbidity values) corresponding to the respective plurality of photosensors can be detected simultaneously in the steps S250, S254, and S262 in FIG. 2B. Therefore, even when the process of FIG. 2B is carried out in parallel for each of a plurality of photosensors, the steps S250, S254, and S262 of FIG. 2B can be carried out only once.

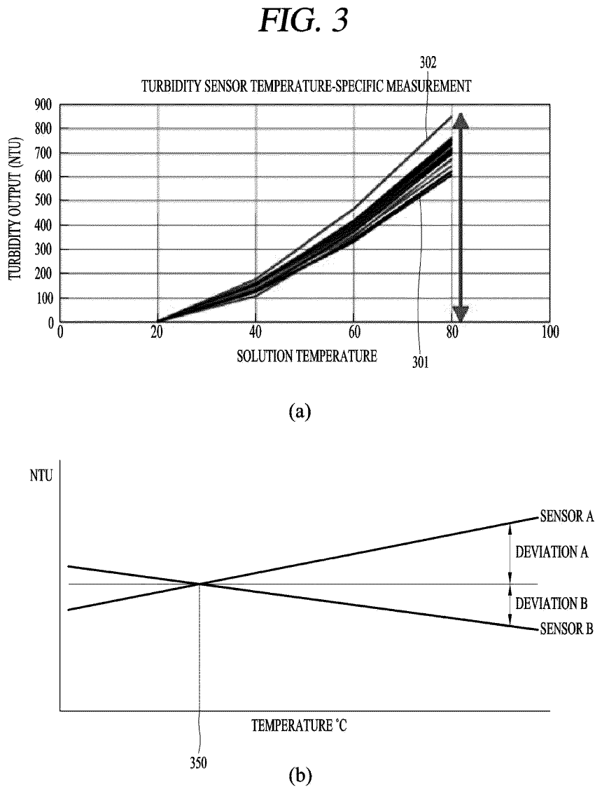

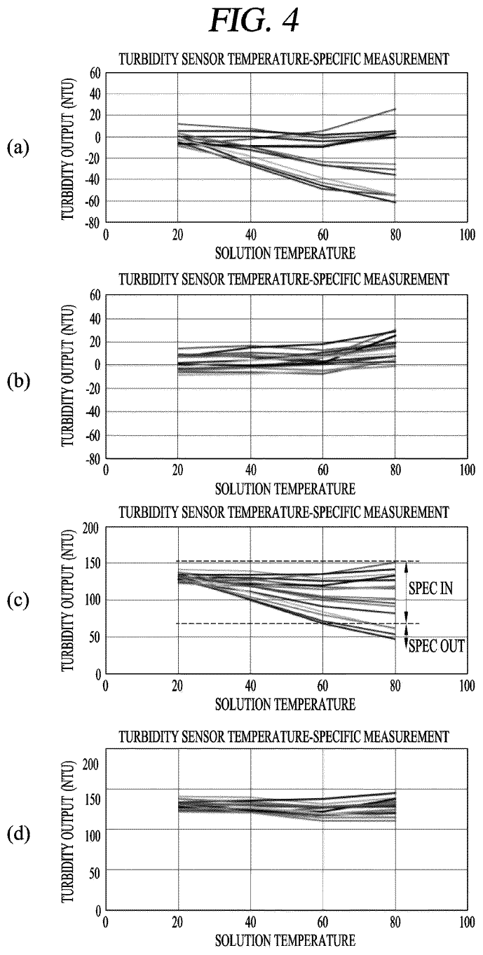

FIG. 3 is exemplary diagrams showing an example of a difference in turbidity detection performance according to an increase in temperature for a plurality of photosensors and a difference in turbidity detection performance deviation between different photosensors. Furthermore, FIG. 4 is exemplary diagrams showing temperature-specific performance deviations of photosensors in a typical case and in a case where the performance deviations are corrected according to an embodiment of the present disclosure.

First, referring to (a) of FIG. 3, (a) of FIG. 3 is an exemplary diagram showing uncorrected NTU values output from 19 photosensors according to a temperature for a test solution (e.g., tap water) having the same turbidity (e.g., 0 NTU) and whose turbidity does not change with temperature for the 19 photosensors.

As shown in (a) of FIG. 3, it is shown that the photosensors can provide different turbidity detection results for the test solution as the temperature increases. Moreover, the photosensors can output NTU values that vary from 0 NTU, from as low as 600 NTU (301) to as high as 800 NTU (302), and can output different NTU values depending on different temperature characteristics. That is, an amount of change in NTU value can vary depending on a temperature change.

Meanwhile, (b) of FIG. 3 shows an example of different turbidity value output deviations for each photosensor depending on temperature characteristics.

As described above, an NTU value according to a temperature change can vary from one another according to a deviation in the temperature characteristics of each photosensor. That is, as shown in (b) of FIG. 3, the same turbidity value can be output at a specific temperature, but when the temperature varies, the output turbidity value can increase or decrease depending on the temperature characteristics. In this case, when a turbidity value being output at a specific temperature is a reference value, there is a problem in that the deviation can increase as the temperature increases from the specific temperature, and due to the deviation, it can be difficult to correct with an average change amount for each temperature characteristic of a plurality of photosensors. Therefore, it is necessary to correct an NTU value being output according to individual temperature characteristics of each of the plurality of photosensors so as to minimize the deviation in the temperature-specific change amount of each photosensor.

FIG. 4 is exemplary diagrams respectively showing temperature-specific performance deviations of photosensors for different turbidities, in a typical case and in a case according to an embodiment of the present disclosure.

First, (a) and (b) of FIG. 4 show examples of cases where a turbidity of the test solution is 0 NTU for 16 photosensors.

(a) of FIG. 4 shows an example of conventional photosensors. In the case of conventional photosensors, it is shown an example in which at a standard temperature of 20 degrees, each photosensor outputs a similar NTU value, but as the temperature increases, an NTU value being output varies significantly due to the temperature characteristics of each photosensor.

Such a performance deviation according to temperature characteristics increases significantly as the temperature increases, and it can be seen that the turbidity performance detection deviation occurs from −60 NTU to 20 NTU even though the test solution is a test solution whose turbidity does not change with temperature when the temperature of the test solution is 80 degrees. Therefore, as shown in (a) of FIG. 4, it can be seen that it is not easy to correct the turbidity value depending on the change characteristics of some photosensors due to the turbidity performance detection deviation. That is, when calibrating by an average value of temperature-specific change amounts for a plurality of photosensors, photosensors whose temperature-specific change amounts follow the average value change amount can be calibrated, but photosensors that do not follow the average value change amount can not be properly calibrated.

In contrast, (b) of FIG. 4 shows an example in which the individual temperature deviation of each photosensor is corrected according to an embodiment of the present disclosure for a test solution (e.g., tap water) having 0 NTU, similar to (a) of FIG. 4. According to an embodiment of the present disclosure, when correction is carried out based on a temperature-specific correction value calculated based on a turbidity difference and a deviation relation for each of a first temperature and a second temperature having a difference of 25 to 30 degrees, the individual temperature deviation of each photosensor can be corrected. Therefore, as shown in (b) of FIG. 4, temperature-specific change amounts in turbidity calculated from a plurality of photosensors can be corrected similarly to one another. That is, since the temperature-specific deviation of each of the plurality of photosensors is small, when calibration is carried out using an average value of the temperature-specific change amounts for the plurality of photosensors, the calibration can be applied to all of the plurality of photosensors. Therefore, each photosensor can output more accurate NTU values depending on the calibration.

Furthermore, (c) and (d) of FIG. 4 show examples of cases where a turbidity of the test solution is 133 NTU for 16 photosensors.

(c) of FIG. 4 shows an example of conventional photosensors. In the case of conventional photosensors, it is shown an example in which at a standard temperature of 20 degrees, each photosensor outputs a similar 133 NTU value, but as the temperature increases, an NTU value being output varies significantly due to the temperature characteristics of each photosensor.

Such a performance deviation according to temperature characteristics increases significantly as the temperature increases, and it can be seen that the turbidity performance detection deviation occurs from 50 NTU to 150 NTU even though the test solution is a test solution whose turbidity does not change with temperature when the temperature of the test solution is 80 degrees.

Therefore, when calibration is carried out by an average value of temperature-specific change amounts for a plurality of photosensors as shown in (c) of FIG. 4 (e.g., calibration that lowers an NTU value being output at 80 degrees by a predetermined value), a photosensor whose temperature-specific change amount follows the average value change amount (spec-in) can be calibrated, but a photosensor that does not follow the average value change amount (spec-out) can actually increase the deviation due to the calibration.

In contrast, (d) of FIG. 4 shows an example in which the individual temperature deviation of each photosensor is corrected according to an embodiment of the present disclosure for a test solution (e.g., tap water) having 133 NTU, similar to (c) of FIG. 4. According to an embodiment of the present disclosure, when correction is carried out based on a temperature-specific correction value calculated based on a turbidity difference and a deviation relation for each of a first temperature and a second temperature having a difference of 25 to 30 degrees, the individual temperature deviation of each photosensor can be corrected.

Therefore, as shown in (d) of FIG. 4, temperature-specific change amounts in turbidity calculated from a plurality of photosensors can be corrected similarly to one another. That is, since the temperature-specific deviation of each of the plurality of photosensors is small, when calibration is carried out using an average value of temperature-specific change amounts for the plurality of photosensors, all of the plurality of photosensors can output more accurate NTU values according to the calibration.

The foregoing present disclosure can be implemented as computer-readable codes on a program-recorded medium. The computer-readable medium includes all kinds of recording devices in which data readable by a computer system is stored. Examples of the computer-readable medium include a hard disk drive (HDD), a solid state disk (SSD), a silicon disk drive (SDD), a ROM, a RAM, a CD-ROM, a magnetic tape, a floppy disk, an optical data storage device and the like, and also include a device implemented in the form of a carrier wave (for example, transmission via the Internet). In addition, the computer can include the control part 100 of the photosensor temperature-specific performance deviation correction apparatus 1 according to an embodiment of the present disclosure.

The detailed description is therefore to be construed in all aspects as illustrative and not restrictive. The scope of the present disclosure should be determined by reasonable interpretation of the appended claims and all changes that come within the equivalent scope of the present disclosure are included in the scope of the present disclosure.

1. A temperature-specific performance deviation correction apparatus for a photosensor, the apparatus comprising:
a sensing part comprising a plurality of photosensors; and
a control part that detects turbidity values at a first temperature and a second temperature for a test solution having a same turbidity from each of the plurality of photosensors, detects a difference between the turbidity values according to the first temperature and the second temperature, calculates temperature-specific turbidity value (NTU) difference estimates for each photosensor based on the detected turbidity value difference, calculates temperature-specific turbidity value correction values for each of the plurality of photosensors by reflecting the temperature-specific turbidity value difference estimates to temperature-specific measurement values measured from each of the plurality of photosensors at each temperature for the test solution having the same turbidity, and corrects the turbidity values of each of the plurality of photosensors according to the calculated temperature-specific turbidity value correction values.

2. The apparatus of claim 1, wherein the control part calculates temperature-specific turbidity value difference estimates for the each photosensor based on a deviation relation that uses a difference between turbidity values according to the first temperature and the second temperature according to the following Equation 1:
$$NTU difference estimate = \frac{Z \times temperature}{30} - \frac{2 \times Z}{3} \qquad [ Equation 1 ]$$
wherein Z is a difference between a first turbidity value detected at the first temperature and a second turbidity value detected at the second temperature.

3. The apparatus of claim 1, wherein the first temperature and the second temperature have a temperature difference between 25 degrees and 30 degrees.

4. The apparatus of claim 1, wherein the test solution is a solution whose turbidity does not change according to a temperature change.

5. The apparatus of claim 1, wherein the control part detects an NTU conversion equation that converts a detection value of a photosensor into a turbidity value (NTU) based on turbidity values detected from the test solution having different turbidities at the same temperature, and detects a difference between a first conversion value obtained by converting a first detection value of a photosensor detected when the test solution is at the first temperature at a same turbidity according to the detected conversion equation and a second conversion value obtained by converting a second detection value of the photosensor detected when the test solution is at the second temperature at the same turbidity according to the detected conversion equation, as a difference between turbidity values according to the first temperature and the second temperature.

6. The apparatus of claim 1, further comprising:
an accommodation part provided with the plurality of photosensors on an inside thereof to accommodate the test solution,
wherein the accommodation part further comprises a heating part capable of heating the test solution.

7. A washing machine comprising:
a sensing part comprising a plurality of photosensors; and
a control part that detects turbidities at a first temperature and a second temperature for a test solution having a same turbidity for each of the plurality of photosensors, detects a turbidity difference according to the first temperature and the second temperature based on a result of the detected turbidities, calculates temperature-specific turbidity value (NTU) difference estimates for each photosensor based on the detected turbidity difference, calculates temperature-specific turbidity value correction values for each of the plurality of photosensors by reflecting the temperature-specific turbidity value difference estimates to temperature-specific measurement values measured from each of the plurality of photosensors at each temperature for the test solution having the same turbidity, and corrects the turbidity values of each of the plurality of photosensors according to the calculated temperature-specific turbidity value correction values.

8. The washing machine of claim 7, wherein the control part calculates temperature-specific turbidity value difference estimates for the each photosensor based on a deviation relation that uses a difference between turbidity values according to the first temperature and the second temperature according to the following Equation 1:
wherein the first temperature and the second temperature have a temperature difference between 25 degrees and 30 degrees.
$$NTU difference estimate = \frac{Z \times temperature}{30} - \frac{2 \times Z}{3} \qquad [ Equation 1 ]$$
wherein Z is a difference between a first turbidity value detected at the first temperature and a second turbidity value detected at the second temperature.

9. The washing machine of claim 7, further comprising:
a laundry accommodation part provided with the plurality of photosensors on an inside thereof to accommodate the test solution.

10. A correction method of correcting temperature-specific performance deviations of a plurality of photosensors for detecting a turbidity of a solution, the method comprising:
detecting, for each of the plurality of photosensors, turbidity values at a first temperature and a second temperature for a test solution having a same turbidity;
calculating a difference between the detected turbidity values;
calculating temperature-specific turbidity value (NTU) difference estimates for each photosensor based on the calculated difference between the turbidity values;
measuring, for the test solution having the same turbidity, turbidity values from each of the plurality of photosensors at each temperature;
calculating, for each of the plurality of photosensors, temperature-specific turbidity value correction values by reflecting the temperature-specific turbidity value difference estimates to the turbidity values measured at each temperature; and
correcting turbidity values of each of the plurality of photosensors according to the calculated temperature-specific turbidity value correction values.

11. The method of claim 10, wherein the detecting of turbidity values at the first temperature and the second temperature comprises:
detecting turbidity values detected from the test solutions having different turbidities at a same temperature;
detecting an NTU conversion equation that converts a detection value of a photosensor into a turbidity value (NTU) based on turbidity values detected from the test solutions having different turbidities;
detecting a first conversion value obtained by converting a first detection value of each photosensor detected when the test solution is at the first temperature at a same turbidity according to the detected conversion equation;
detecting a second conversion value obtained by converting a second detection value of each photosensor detected when the test solution is at the second temperature at the same turbidity according to the detected conversion equation; and
detecting, for each of the plurality of photosensors, a difference between the first conversion value and the second conversion value as a difference between turbidity values according to the first temperature and the second temperature.

12. The method of claim 11, wherein the detecting of a difference between the first conversion value and the second conversion value as a difference between the turbidity values comprises:
detecting photosensors, from among the plurality of photosensors, having a difference between the first conversion value and the second conversion value within a preset range; and
excluding the detected photosensors from those for temperature-specific turbidity value correction.

13. The method of claim 10, wherein the calculating of the temperature-specific turbidity value (NTU) difference estimates comprises:
calculating temperature-specific turbidity value difference estimates for the each photosensor based on a deviation relation that uses a difference between turbidity values according to the first temperature and the second temperature according to the following Equation 1:
$$NTU difference estimate = \frac{Z \times temperature}{30} - \frac{2 \times Z}{3} \qquad [ Equation 1 ]$$
wherein Z is a difference between a first turbidity value detected at the first temperature and a second turbidity value detected at the second temperature.

14. The method of claim 10, wherein the first temperature and the second temperature have a temperature difference between 25 degrees and 30 degrees.

15. The method of claim 10, wherein the test solution is a solution whose turbidity does not change according to a temperature change.
